(19) United States
(12) Patent Application Publication
(10) Pub. No.: US 2009/0053181 A1
(43) Pub. Date: Feb. 26, 2009

(54) Modulation of Cells

(75) Inventor: Palle Serup, Copenhagen (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsværd (DK)

(21) Appl. No.: 11/916,544

(22) PCT Filed: May 24, 2006
(86) PCT No.: PCT/EP2006/062601

§ 371 Date: Jan. 7, 2008

Related U.S. Application Data
(60) Provisional application No. 60/691,168, filed on Jun. 16, 2005.

(30) Foreign Application Priority Data
Jun. 13, 2005 (EP) 05105149.8

(51) Int. Cl. A61K 35/12; C12N 5/06; C12Q 1/02; C12Q 1/54; A61P 3/10

(52) U.S. Cl. 424/93.7; 435/373; 435/29; 435/14; 435/325

(57) ABSTRACT

The present invention is directed to methods which can be used to test agents for their ability 5 to modulate cell proliferation, differentiation and other biological activities of target cells such as stem cells. In addition, the invention is also directed to methods for modulating a biological activity of target cells in vitro, to the cells whose biological activity is modulated by such methods, to the cells that may be produced from target cells by such methods, and to the use of such cells for transplantation, transfusion and other purposes.

### FIELD OF THE INVENTION

This invention relates to methods for modulating a biologically activity of target cells in cell culture and for testing candidate agents for their ability to modulate a biologically activity of target cells in cell culture.

The present invention is directed to methods which can be used to test agents for their ability to modulate cell proliferation, differentiation and other biological activities of target cells such as stem cells. In addition, the invention is also directed to methods for modulating a biological activity of target cells in vitro, to the cells whose biological activity is modulated by such methods, to the cells that may be produced from target cells in such methods, and to the use of such cells for transplantation, transfusion and other purposes.

### BACKGROUND OF THE INVENTION

There is considerable effort devoted to the development of assay systems for identifying agents that are able to modulate cellular activities such as cell growth and differentiation and a number of such assay systems are currently in use to identify such agents and to use such agents, once identified, to modify cellular activities of target cells.

The development of assays to identify factors that can stimulate the differentiation of stem cells has been one area of significant interest in view of the ability of stem cells to differentiate under the right conditions to the many different cell types that make up an organism. Hence, stem cells may be a potentially renewable source of cells that can differentiate into a variety of cell types useful for treating diseases such as Parkinson's and type 1 diabetes.

Stem cells are cells that have the ability to self replicate for indefinite periods and have the potential to develop into mature cells that have specialized functions such as heart cells, nerve cells and pancreatic beta cells. There are several types or sources of stem cells.

Embryonic stem (ES) cells are stem cells derived from the inner mass cells of a blastocyte and are pluripotent; i.e., they can differentiate into cells derived from all three primary germ layers: ectoderm, endoderm or mesoderm.

Adult stem cells are undifferentiated cells that reproduce daily to provide certain specialized cells. Adult stem cells have been identified throughout the body including in bone marrow, peripheral blood, brain, spinal cord, liver and pancreas and they have more limited potential than ES cells. Typically, adult stem cells are multipotent cells committed to differentiate into cells that contribute to the function of the tissue from which they originated. However, adult stem cells have been identified with the potential to differentiate into specialized cells of unrelated tissues, including cells derived from a different embryonic germ layer, under certain conditions.

In designing assays to determine under what conditions stem cells can be induced to differentiate to cells of a desired lineage, it is desirable to identify the right molecule, or more likely, the right combination of molecules, that will support the self-renewal of undifferentiated cells in culture and stimulate them to commit to the desired cell lineage.

Prior to the present invention, methods for identifying the molecule or combination of molecules that could stimulate the differentiation of stem cells to a desired cell lineage relied on micromanipulation, which is time and labor consuming, to establish 1) a continuous gradient for a single molecule or 2) discrete (non-continuous) concentrations of more than one molecule through the use of chambers each containing different concentrations of the molecules.

### SUMMARY OF THE INVENTION

The present invention provides methods for producing a continuous concentration gradient of one or more agents of interest in a cell culture.

In one embodiment, the method comprises culturing cells that recombinantly express at least one cell adhesion molecule and one or more agents of interest under conditions sufficient to allow the cells expressing the cell adhesion molecule(s) to form self-assembling aggregates and secrete the recombinantly expressed agent(s) into the cell culture medium thereby producing a continuous concentration gradient of the agent(s) in the cell culture medium.

In another embodiment, called the gravity assisted method, cells that recombinantly express one or more agents of interest are placed as hanging drops on the lid of a container such as a Petri dish under conditions sufficient that the cells form self-assembling aggregates which, upon displacement from the container into a culture medium, secrete the recombinantly expressed agent(s) into the cell culture medium thereby producing a continuous concentration gradient of the agent(s).

In the methods of the invention disclosed herein, cells that recombinantly express one or more agents of interest and optionally at least one cell adhesion molecule are known as “effector cells” and where the agent(s) of interest expressed by the effector cells are intended to induce a response in the target cells, such cells may also be referred to herein as “inducer cells”. It is to be understood that in the gravity assisted methods for producing self-assembled aggregates as described herein, it is not necessary that the effector cells recombinantly express cell adhesion molecule(s).

The present invention also provides methods for modulating a biological activity of target cells.

In one embodiment, the method for modulating a biological activity of target cells comprises:
A) exposing a cell culture containing the target cells to effector cells that recombinantly express at least one cell adhesion molecule and at least one agent where expression of the agent(s) by the effector cells produces a concentration gradient of agent(s) in the cell culture medium; and
B) culturing the target cells in the presence of said effector cells for a time sufficient to permit the biological activity of said target cells to be modulated by said agent(s) in the cell culture medium.

In another embodiment, the method for modulating a biological activity of target cells comprises:
A) placing target cells and effector cells that recombinantly express at least one agent of interest as hanging drops on the lid of a container such as a Petri dish under conditions sufficient that the target and effector cells form self-assembling aggregates where expression of the agent(s) by said effector cells in said aggregates, upon displacement of said aggregates from the container into a culture medium, produces a continuous concentration gradient of the agent(s) in the cell culture medium; and
B) culturing the self-assembled aggregates for a time sufficient to permit the biological activity of the target cells in said aggregates to be modulated by said concentration gradient of the agent(s) in the cell culture medium.

The present invention further provides methods for evaluating a candidate agent for its ability to modulate a biological activity of target cells.

In one embodiment, the method for evaluating a candidate agent for its ability to modulate a biological activity of target cells comprises:
A) exposing a cell culture containing the target cells to effector cells that recombinantly express at least one cell adhesion molecule and at least one candidate agent where expression of the candidate agent(s) by the effector cells produces a concentration gradient of candidate agent(s) in the cell culture medium; and
B) detecting or measuring the biological activity of the target cells, wherein the detection or measurement of a change of the biological activity of the target cells in the presence of the candidate agent(s) indicates that the candidate agent(s) present in the cell culture medium is capable of modulating the biological activity of the target cells.

In another embodiment, the method for evaluating a candidate agent for its ability to modulate a biological activity of target cells comprises:
A) placing target cells and effector cells that recombinantly express one or more candidate agents as hanging drops on the lid of a container such as a Petri dish under conditions sufficient that the target and effector cells form self-assembling aggregates where, upon displacement of said aggregates from the container into a culture medium, expression of the agent(s) by said effector cells in said aggregates produces a continuous concentration gradient of the agent(s) in the cell culture medium;
B) culturing said aggregates for a time sufficient to permit the biological activity of the target cells in said aggregates to be modulated by said concentration gradient of the agent(s) in the cell culture medium; and
C) detecting or measuring the biological activity of the target cells, wherein the detection or measurement of a change of the biological activity of the target cells in the presence of the candidate agent(s) indicates that the candidate agent(s) present in the cell culture medium is capable of modulating the biological activity of the target cells.

In another embodiment, the target cells in the above methods are stem cells and the biological activity to be modulated is differentiation of the stem cells to a cell of a desired lineage.

The present invention also relates to effector cells used in the methods of the invention and in particular to effector cells that have been transfected with nucleic acid sequences encoding agents and/or specific combinations of cell adhesion molecules and agents.

The present invention also relates to target cells that have been exposed to agents according to the methods of the invention, to cells that have been generated from target cells exposed to agents according to the methods of the invention, to pharmaceutical compositions comprising such cells, and to method of use of such cells.

### BRIEF DESCRIPTION OF THE FIGURES

FIGS. 1A-D illustrate gravity assisted assembly of cell aggregates composed of “inducer cells” (producing growth factor X) and target cells (ES) where according to Stoke's Law, aggregates of cells will sediment faster than single cells.

FIG. 1A shows a protocol for assembly of inducer and target cells (the actual number of inducer and target cells may vary).

FIG. 1B presents a drawing of the assembly process (not to scale).

FIG. 1C presents a schematic drawing of a single inducer/target cell assembly where the inducer cells are shown in red and the target cells are shown in light blue. The increasing distance from the inducer cells is indicated by the solid arrow and target cells located close but equidistant to the inducer cells are indicated by broken line 1, while target cells located at (equidistant) intermediate and distant positions relative to the inducer cells are indicated by broken lines 2 and 3, respectively.

FIG. 1D presents a diagram showing the change in concentration of X ([X]) that the target cells are exposed to as a function of distance from the source of X (inducer cells). Three discrete distances from the inducer cells (corresponding to 1, 2, and 3 in FIG. 1C) and the corresponding concentration of X (1, 2, and 3) that the target cells will be exposed to are indicated. Target cells that are distributed along broken line 1 (in FIG. 1C) will be exposed to higher a concentration of X than target cells distributed along broken line 2 (in FIG. 1C), which again will be exposed to a higher concentration of X than target cells that are distributed along broken line 3 (in FIG. 1C). Given that the target cells have the capacity to respond differently to different concentrations of X, target cells distributed along broken line 1 (in FIG. 1C) will respond differently than target cells distributed along broken line 2 (in FIG. 1C), which again will respond differently than target cells that are distributed along broken line 3 (in FIG. 1C). The actual time needed for the cells to aggregate can vary between different cells. In some instances the cells may require 24 hours to aggregate in each step, changing the overall time in hanging drops to 48 hours prior to further culture in dishes.

FIG. 2 illustrates a procedure for generation of fluorescently tagged cadherin expressing effector cells that are sensitive to Gancyclovir. “E-cad” indicates E-cadherin, “N-cad” indicates N-cadherin, “R-cad” indicates R-cadherin, “PGK-hsv tk” indicates a plasmid encoding the Herpes simplex thymidine kinase gene (hsv tk) under control of the phosphoglyceratekinase (PGK) promoter, and HAT indicates hypoxanthine, aminopterine, thymidine). In the procedure illustrated in FIG. 2, thymidine kinase negative (tk−) mouse L-cells the are used as the effector cells and the initial stable transfection needed to introduce the cadherin plasmids can be made by co-transfection of a plasmid encoding the Herpes simplex thymidine kinase gene (hsv tk) under control of the phosphoglyceratekinase (PGK) promoter where expression of hsv tk confers HAT (hypoxanthine, aminopterine, thymidine) resistance to tk-cells. hsv tk+ clones can thus be selected in HAT medium and the resulting clones can after expansion be screened for fluorescent protein expression by fluorescence microscopy followed by screening for cadherin expression by western blotting and immunocytochemistry. The resulting isolated clones will in addition to the cadherin and fluorescent protein expression have acquired sensitivity to Gancyclovir due to the expression of hsv tk.

FIGS. 3A-3D illustrate cadherin mediated assembly of cell aggregates composed of “inducer cells” (producing growth factor X) and target cells (ES). Two cell populations that express different cadherins will sort themselves according to the cadherin expression such that cells that express a given cadherin will aggregate with other cells that express the same cadherin but avoid aggregation with cells that express a different cadherin.

FIG. 3A illustrates a protocol for cadherin mediated assembly of inducer and target cells (the actual number of inducer and target cells may vary).

FIG. 3B represents a drawing of the assembly process (not to scale).

FIG. 3C presents a schematic drawing of a single inducer/target cell assembly where the inducer cells are shown in green and the target cells are shown in light blue, the increasing distance from the inducer cells is indicated by the solid arrow and target cells located close but equidistant to the inducer cells are indicated by broken line 1, while target cells located at (equidistant) intermediate and distant positions relative to the inducer cells are indicated by broken lines 2 and 3, respectively.

FIG. 3D presents a diagram showing the change in concentration of X ([X]) that the target cells are exposed to as a function of distance from the source of X (inducer cells). Three discrete distances from the inducer cells (corresponding to 1, 2, and 3 in FIG. 3C) and the corresponding concentration of X (1,2, and 3) that the target cells will be exposed to are indicated. Target cells that are distributed along broken line 1 (in FIG. 3C) will be exposed to higher a concentration of X than target cells distributed along broken line 2 (in FIG. 3C), which again will be exposed to a higher concentration of X than target cells that are distributed along broken line 3 (in FIG. 3C). Given that the target cells have the capacity to respond differently to different concentrations of X, target cells distributed along broken line 1 (in FIG. 3C) will respond differently than target cells distributed along broken line 2 (in FIG. 3C), which again will respond differently than target cells that are distributed along broken line 3 (in FIG. 3C). As long as the target cells express E-cadherin, the inducer cells may be express any classical cadherin different from E-cadherin. For example, expression of either N-cadherin, R-cadherin, P-cadherin, or L-cadherin in the inducer cells will result in the same outcome. If the target cells express another cadherin than E-cadherin the inducer cells can express any cadherin except that expressed by the target cells.

FIGS. 4A-4H show cadherin mediated assembly of cell aggregates composed of two different “inducer cells” (producing growth factors X and Y, respectively) and target cells (ES).

FIG. 4A presents a protocol for cadherin mediated assembly of two distinct inducer cells and target cells (the actual number of inducer and target cells may vary).

FIG. 4B presents a drawing of the assembly process (not to scale).

FIG. 4C presents a schematic drawing of the spatial distribution of inducer cells X and Y in relation to target cells for the purpose of obtaining opposing gradients of two different growth factors.

FIG. 4D presents a diagram showing the change in concentration of X ([X]) and Y ([Y]) that the target cells are exposed to as a function of distance from the source of X and Y, respectively (the inducer cells X and Y).

FIG. 4E shows a possible spatial distribution of inducer and target cells assembled according to the protocol shown in FIG. 4A.

FIG. 4F presents a diagram showing the change in concentration of X ([X]) and Y ([Y]) that the target cells are exposed to as a function of the distance from the source of X and Y, respectively (the inducer cells X and Y) along the axes depicted in FIGS. 4C (1) and 4E (2).

FIG. 4G presents a schematic representation of axes along which the change in concentration of growth factors X and Y that target cells will be exposed to is following the change depicted in FIG. 4H.

FIG. 4H presents a diagram showing the change in concentration of X ([X]) and Y ([Y]) that the target cells are exposed to as a function of the distance from the source of X and Y, respectively (the inducer cells X and Y) along the axes depicted in FIG. 4H.

FIG. 5 shows assembly of self-aggregates according to the protocol shown in FIG. 4 in a situation where E-cad expression (indicated as E⁺⁺ for ES target cells and as E⁺⁺ for L inducer cells) is higher in inducer cells (i.e. L cells in the Figure) than in target cells (indicated in the Figure as ES cells). As illustrated in FIG. 5, in a scenario where E-cad expression is higher in inducer cells than in target cells, aggregates will contain the inducer cells surrounded by the target cells.

FIG. 6 shows assembly of self-aggregates according to the protocol shown in FIG. 4 in a situation where E-cad expression (indicated as E⁺ for L inducer cells and as E⁺⁺ for ES target cells) is lower in inducer cells (i.e. L cells in the Figure) than in target cells (indicated in the Figure as ES cells). As illustrated in FIG. 6, in a scenario where E-cad expression is lower in inducer cells than in target cells, aggregates will contain the inducer cells surrounded by the target cells.

FIG. 7 shows assembly of self-aggregates according to the protocol shown in FIG. 4 in a situation where E-cad expression (indicated as E⁺ for L inducer cells and as E⁺⁺ for ES target cells) in target cells is equal to that of the inducer cells. As illustrated in FIG. 7, in a scenario where E-cad expression in target cells is equal to that of the inducer cells, aggregates will contain the target cells intermingled with the inducer cells.

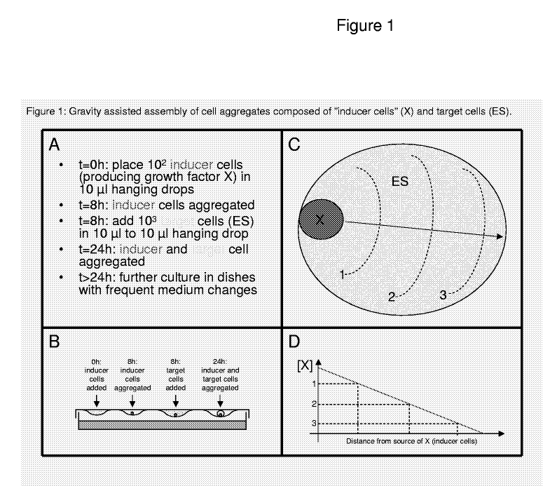

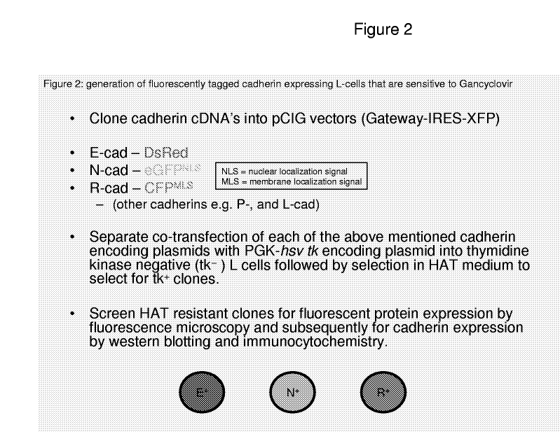

### DESCRIPTION OF THE INVENTION

The present invention provides methods for modulating a biological activity of target cells. In one embodiment, the method comprises:
A) exposing a cell culture medium containing the target cells to effector cells that recombinantly express at least one cell adhesion molecule and at least one agent where expression of the agent(s) by the effector cells produces a concentration gradient of agent(s) in the cell culture medium; and
B) culturing the target cells in the presence of said effector cells for a time sufficient to permit the biological activity of the target cells to be modulated by the agent(s) in the cell culture medium.

In this method, the agent or agents to be recombinantly expressed by the effector cells are agents that have previously been identified and/or are known as molecules capable of modulating the biological activity of the target cell that one desires to modulate in this method.

The present invention also provides methods for evaluating a candidate agent (by “candidate agent” is simply meant an agent that prior to testing in the present method, had not been identified as an agent capable of modulating the biological activity of the target cell that is to be measured in the method) for its ability to modulate a biological activity of target cells. In one embodiment, this method comprises:
A) exposing a cell culture medium containing the target cells to effector cells that recombinantly express at least one cell adhesion molecule and at least one candidate agent where expression of the agent(s) by the effector cells produces a concentration gradient of candidate agent(s) in the cell culture medium; and
B) detecting or measuring the biological activity of the target cells, wherein the detection or measurement of a change of the biological activity of the target cells in the presence of the candidate agent(s) indicates that the candidate agent(s) in the cell culture medium is capable of modulating the biological activity of the target cells.

While the concentration gradient of agent(s) produced in the above methods is a continuous one, the “shape” of the concentration gradient can be altered in a number of ways including 1) varying the degree of intermingling of target cells and effector cells, 2) using opposing agonists/antagonists (the “source/sink” concept) as the agents recombinantly expressed by effector cells and/or 3) varying the ratio of the target cells to effector cells where in 3), the shape of the gradient will not change as one varies the ratio of target cells to effector cells but the minimum and maximum values of the gradient will vary.

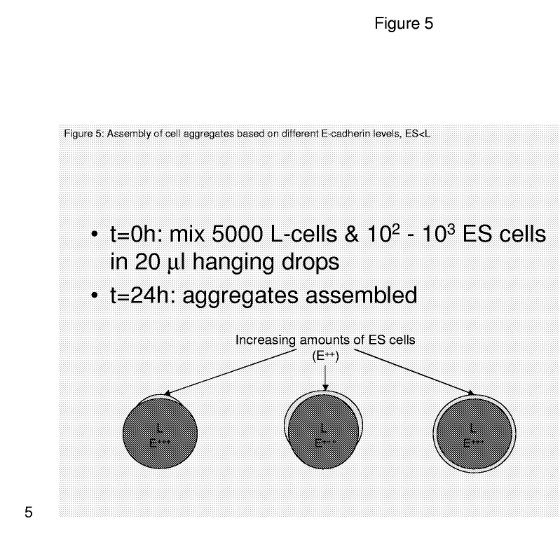

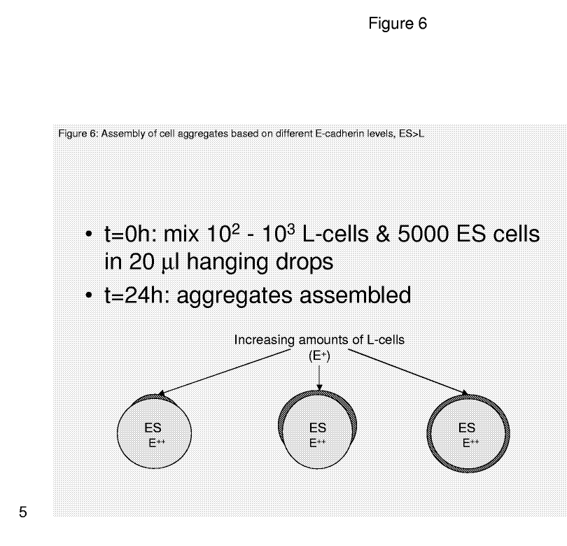

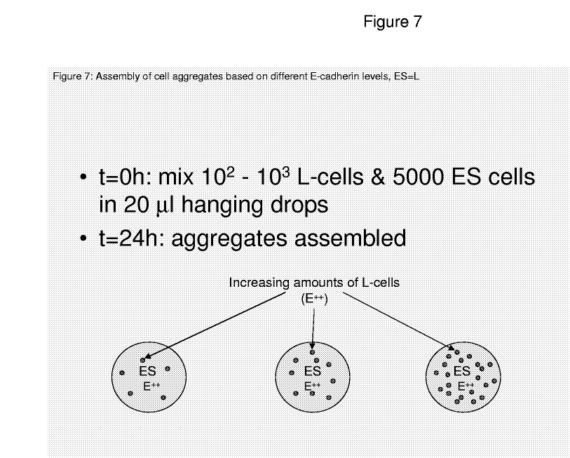

With respect to the degree of intermingling of target cells and effector cells, this is dependent on whether the target and effector cells express the same cell adhesion molecules and on the level of expression of those molecules. For example, if the target cells express high levels of cell adhesion molecule A and the effector cells express low levels of the cell adhesion molecule A, then the target cells will cluster together in the center and the effector cells will be on the periphery of the cluster of the target cells (see FIG. 6). Conversely, the reverse arrangement of effector and target cells will occur if the effector cells express high levels of cell adhesion molecule A and the target cells express low levels of the cell adhesion molecule A (see FIG. 5). Alternatively, if both the effector and target cells express the same cell adhesion molecule at similar concentrations, one will get intermingling of the target and effector cells (see FIG. 7).

In another embodiment, if the target cells express cell adhesion molecule A and the effector cells express cell adhesion molecule B, then the target and effector cells will also not intermingle with each other.

Of course, whether one desires to have intermingling between target and effector cells will depend on what type of concentration gradient one desires to produce in the culture medium for the agent being tested. For example, if one wants a diffuse even exposure of target cells to the agent recombinantly expressed by the effector cells then intermingling between the target and effector cells would be a desirable outcome. Alternatively, if one wants exposure of the target cells to a steeper concentration gradient of the agent, then intermingling between the target and effector cells would not be desirable. In this regard, the concentration of agent “seen” by the target cell will depend on the distance to the effector cells such that the closer the target cell is to the effector cell the higher the concentration of agent the target cell will see.

In another embodiment, the shape of the concentration gradient of agent can be changed by having one population of effector cells as a “source” of agent and another population of effector cells as a “sink” for the agent where the concept of a source and a sink is analogous to how the embryo often sets up gradients [for example, the dorsal-ventral patterning of the neural tube that uses dorsally localized BMP and ventrally localized Chordin (a BMP antagonist) in addition to the ventrally located Shh]. For example, one could enable the population of effector cells expressing an antagonist (i.e., the “sink”) to intermingle with the target cells and the population of effector cells expressing an agonist (i.e., the “source”) not to intermingle with the target cells or vice versa by the choice of cell adhesion molecule that is transfected into each population of effector cells and by the type of cell adhesion molecule that was expressed in the target cells.

A specific example of how the “source/sink” concept can be utilized to produce a concentration gradient of agent is to transfect one population of effector cells with the RALDH2 gene which encodes a retinoic acid (RA) producing enzyme that uses vitamin A as a precursor to produce RA that diffuses away from the effector cells and to transfect a second population of effector cells with one or more CYP26 isoforms which are RA degrading enzymes of the cytochrome P450 family.

By “effector cell” as used herein is meant any cell in which an agent and optionally a cell adhesion molecule can be recombinantly expressed. When the effector cell is a cell in-which at least one agent and at least one cell adhesion molecule are to be expressed then in one embodiment, the effector cell is a cell or cell line that exhibits little or no endogenous expression of the cell adhesion molecule which the effector cell is to recombinantly express. In another embodiment, the effector cell is derived from the same species as the target cell is obtained from. Thus, for example, if the target cells were adult stem cells obtained from a human, the effector cells would be cells derived from a human.

In yet another embodiment, where the cell adhesion molecule to be recombinantly expressed in the effector cell is a member of the cadherin superfamily, the effector cells may be selected from mesenchymal cell lines including, but not limited to fibroblast cell lines such as L cells or CHO cells.

By “cell adhesion molecule” as used herein is meant any cell membrane bound protein or fragment thereof that mediates intercellular recognition and adhesion such that cells expressing the same cell adhesion molecule will self-aggregate. In one embodiment, the cell adhesion molecules will be molecules that are not involved in cell signalling. In the methods of the invention, it is preferred that the adhesion molecules to be recombinantly expressed in the effector cells are from the same species.

Examples of cell adhesion molecules that may be used in the methods of the present invention include, but are not limited to, members of the cadherin superfamily or the Eph receptor family (named for its expression in an eryhropoietin-producing human hepatocellular carcinoma cell line).

The cadherins are single-pass transmembrane proteins that mediate calcium dependent cell-cell adhesion and the cadherin superfamily consist of several subfamilies comprising the classical cadherins, desmosomal cadherins and the protocadherins and nucleic acid sequences encoding many of these members of the cadherin superfamily are known [see Table 2 of Yagi et al (2000) Genes and Development, 14:1169-1180, the contents of which is hereby incorporated by reference] The members of the three families differ in the number of tandem repeats they possess with the classical cadherins having 4 tandem repeats, the desmosomal cadherins having 5 and the protocadherins having either 6 or 7.

The classical cadherins are designated CDH1 through CDH22 with the most well characterized cadherins CDH1 through CDH4 being also commonly known as the E-(epithelial), N-(neural), P-(placental) and R-(retinal) cadherins. In one embodiment, the cell adhesion molecules that may be used in the methods of the present invention are selected from the classical cadherins and in another embodiment, are selected from the E-, N-, P- and R-cadherins.

As the cadherin molecules have three major regions; an extracellular region that mediates cell specific adhesion, a transmembrane domain that spans the cell membrane and a cytoplasmic domain that extends into the cell, it is to be understood that fragments of cadherins or chimeric cadherins created by fusing together different regions of two cadherins could also be utilized as cell adhesion molecules in the present invention.

In yet another embodiment, the cell adhesion molecules that may be used in the methods of the present invention are selected from the protocadherin subfamily where examples of such protocadherins includes, but is not limited to, CNRs, Pcdh alpha, beta or gamma and Pcdh7, 8, 9 or 11.

In another embodiment, the cell adhesion molecule may be an Eph receptor where the Eph receptors are divided into two groups, the EphA and EphB receptors, on the basis of the relatedness of their extracellular domain sequences and the ability of the receptors to bind to the ephrin A or ephrin B ligands. There are at least 9 members of the EphA group of receptors designated EphA1 through EphA9 and at least 6 members of the EphB group of receptors designated EphB1 through EphB6 and full length sequences of each of these receptors are known.

Of course, it is understood that an effector cell may be transfected by nucleic acid molecules encoding one or more cell adhesion molecules where by “transfected” is meant any method for introducing nucleic acid molecules into cells where such methods include, but are not limited to, lipofection, DEAE-dextran mediated transfection, calcium phosphate precipitation, retroviral delivery and electroporation. Recombinant expression of a nucleic acid molecule in an effector cell may be achieved by operably linking a nucleic acid molecule encoding a cell adhesion molecule to a promoter and any other sequences known to those of skill in the art as being sufficient to direct recombinant expression of the cell adhesion molecule in the transfected effector cell. Of course, it is understood that the promoters to be utilized to express the nucleic acid sequences encoding the cell adhesion molecules (and agents) of the invention may be constitutive promoters or inducible promoters where such promoters and their sequences are known to those of ordinary skill in the art.

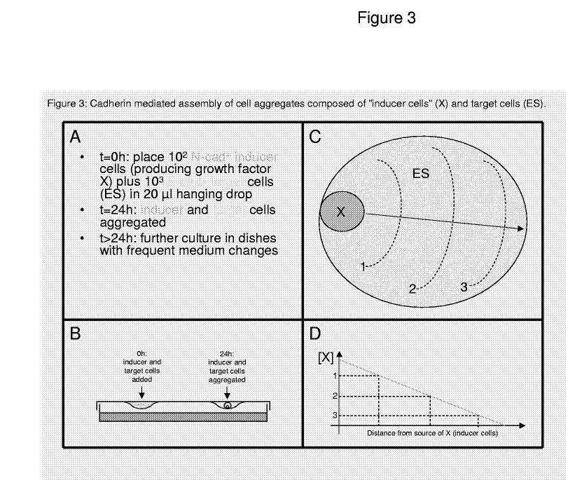

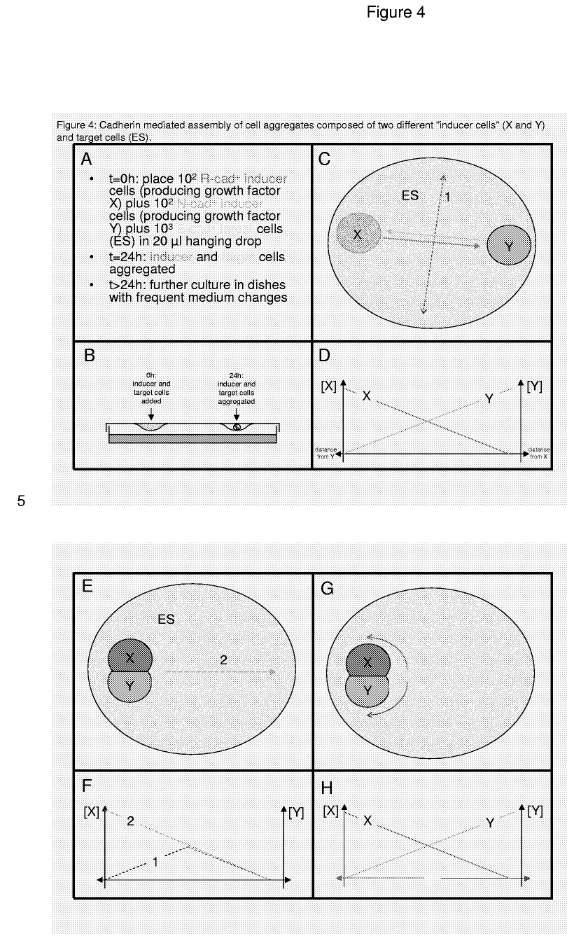

In one embodiment, one may choose to recombinantly express a single type of cell adhesion molecule in each effector cell. Thus, for example, one might recombinantly express E-cadherin molecules in one population of effector cells, N-cadherin molecules in another population of effector cells and so on. Where one wishes to track different populations of effector cells; for example, where one produces different populations of effector cells that recombinantly express different cell adhesion molecules it may also be desirable to transfect the effector cell populations with sequences encoding different fluorescent proteins. Examples of commercially available fluorescent protein markers are known to those of ordinary skill in the art and include those described in FIG. 3 as well as vectors sold by Evrogen under the names Cop-Green (green color), Phi-Yellow (yellow color) and HcRed Tandem (far red color).

Thus, for example, one might transfect a first population of effector with a nucleic acid sequence encoding E-cadherin and with a sequence encoding a green fluorescent protein and a second population of effector cells with a sequence encoding N-cadherin and a sequence encoding a red fluorescent protein.

The present invention therefore also relates to effector cells used in the methods of the invention and in particular to effector cells that have been transfected with nucleic acid sequences encoding specific combinations of sequences encoding cell adhesion molecules, agents and optionally fluorescent marker proteins.

It is to be understood that the type of cell adhesion molecule one wishes to recombinantly express in an effector cell and the level of expression of such a recombinantly expressed cell adhesion molecule that one desires to obtain may be determined by factors such as those described above including the type of cell adhesion molecule endogenously expressed in the target cells and the level of expression of that molecule in the target cells, the degree of intermingling between effector cells and target cells that one desires, and/or the shape of concentration gradient of the agent or candidate agent that one desires the target cells to be exposed to.

By “target cell” as used throughout this application is meant any cell or cell line whose biological activity one might desire to modulate via the methods of the invention. Such target cells may be obtained from humans, other primates, mice, rats, pigs, cows, non-mammals such as chickens and any species that is used as a model system and/or is of commercial interest.

For example, in one embodiment of the invention, the target cell may be any type of stem or progenitor cells that have the potential to differentiate into more specialized or committed progeny cells. In such an embodiment, the stem cell to be chosen may be an embryonic stem cell or an adult stem cell and where adult stem cells are to be used, the choice of adult stem cell to be utilized in the methods of the invention will depend on the type of specialized cells one wishes to produce. For example, if one wanted to produce specialized cells of the pancreas, one might choose intestinal stem cells as the adult stem cells to be used as target cells. Methods for obtaining embryonic stem cells and adult stem cells are known in the art (see, for example, U.S. Pat. No. 5,843,780, which is hereby incorporated by reference and which describes the isolation of primate embryonic stem cells).

For embryonic stem cells, since these cells are pluripotent and can give rise to cells derived from all three embryonic germ layers (mesoderm, ectoderm and endoderm) where it is understood that cells derived from the mesoderm can include cells of the bone marrow, adrenal cortex, lymphatic tissue, skeletal, smooth and cardiac muscle, connective tissue such as bone and cartilage, and those of the heart and blood vessels; cells derived from the ectoderm can include cells of the skin, neural tissue, pituitary gland, eyes, ears and connective tissue of the neck and face; and cells derived from the endoderm can include cells of the lining of the respiratory and gastrointestinal (GI) tract, of the GI organs such as liver and pancreas and cells of the thymus, thyroid and lung; one would recognize that the type of cells produced from an ES cell would depend on the type of agent(s) such ES cells were exposed to in the methods of the invention.

In another embodiment, target cells may be selected from dopaminergic neurons, beta cells, hepatocytes and cardiomyocytes.

Of course, it is understood that the choice of agent(s) to use in the methods of the invention will depend on the outcome you want to achieve using the methods of the invention. For example, if one wanted to produce neural cells from ES cells, one would expose the ES cells to a different set of agents than one would expose ES cells to if one wanted to produce beta cells.

In another embodiment, the target cell may be a progenitor cell such as a pancreatic progenitor cell where such cells can be identified by the presence or absence of particular antigens. For example, PDX1+NR×6.1+p48−PTF1+ cells (where the “+” sign is understood by those of ordinary skill in the art to indicate that the cell is positive for expression of the protein or peptide preceding the + sign) are multipotent pancreatic progenitor cells.

In another embodiment, the target cell may be a cell whose proliferation one wants to modulate such as a beta cell, a neural cell, a heart cell or a cancer cell.

In another embodiment, the target cell may be any cell which has, or is capable of having, an enzyme activity one wishes to modulate. For example, enzymes of the cytochrome P450 family are enzymes whose activity is known to be induced or suppressed by exposure to different agents and thus cells expressing cytochrome P450 enzymes could be target cells in the methods of the invention.

In a further embodiment, the target cell may be a cell that contains receptors whose ligand binding and/or cell signalling one wants to modulate.

In yet another embodiment, the target cell may be any cell that exhibits or is capable of exhibiting a chemotactic response to an agent where such cells include, but are not limited to, monocytes, neutrophils and macrophages.

By “biological activity” of the target cell that is to be modulated in the methods of the invention is meant any biological activity that can be carried out by or in a target cell. Of course, it is understood that the type of biological activity to be modulated will depend on a number of factors including the type target cell and the type of agents that are utilized in the methods of the invention. Examples of biological activities that can be modulated in the methods of the invention include, but are not limited to, differentiation of the target cells to cells of a desired lineage, proliferation of the target cells (by “proliferation” is meant an increase in cell number or cell size), apoptosis, chemotaxis, receptor binding activity, and enzyme activities.

In one embodiment, the biological activity of the target cell is modulated (i.e., increased or decreased) by an agent(s) in the methods of the invention by at least 20% relative to the biological activity of a target cell that was not exposed to the agent(s).

In another embodiment, the biological activity of the target cell is modulated by an agent(s) in the method of the invention by at least 40% relative to the biological activity of a target cell that was not exposed to the agent(s).

In a further embodiment, the biological activity of the target cell is modulated by an agent(s) in the method of the invention by at least 60% relative to the biological activity of a target cell that was not exposed to the agent(s).

In yet another embodiment, the biological activity of the target cell is modulated by an agent(s) in the method of the invention by at least 75% relative to the biological activity of a target cell that was not exposed to the agent(s).

In another embodiment, the biological activity of the target cell is modulated by an agent(s) in the method of the invention by at least 90% relative to the biological activity of a target cell that was not exposed to the agent(s).

In yet a further embodiment, where the biological activity of the target cell that is to be modulated in the methods of the invention is differentiation of the target cell where the word “differentiation” is well known in the art and is intended to cover the potential of any stem or progenitor cells to produce more specialized or committed progeny cells, preferably, the differentiation is increased by at least 1.5 fold, more preferably by 3 fold and most preferably by at least 5 fold relative to the differentiation of a target cell that was not exposed to the agent.

By “modulating” or “modulated” as used herein is meant increasing or decreasing the biological activity of a target cell. It will be understood that the degree of modulation of a biological activity will depend, aside from the agent(s) used in the methods of the invention, on the biological activity to be modulated by the agent(s) and the particular assay used to measure that biological activity but that one skilled in the art can determine a statistically significant change in the biological activity of the target cell that either constitutes an increase or decrease in the biological activity of the target cell that is being measured or that identifies a candidate agent(s) as an agent(s) that increases or decreases the biological activity of the target cell that is being measured.

Of course, to modulate the biological activity of a target cell in the methods of the present invention, one must expose the target cell to one or more agents where by “agent” is meant a naturally occurring or artificially derived molecule that can modulate a biological response by or in a target cell.

By “naturally occurring molecules” is meant molecules that have been found in vivo in a biological system to directly modulate a biological activity by or in the target cell or to indirectly modulate a biological activity by or in a target cell by for example, synthesizing or producing a molecule that modulates the biological activity by or in the target cell. Examples of such molecules include, but are not limited to, proteins or peptides such as hormones, growth factors, cytokines, chemoattractants and enzymes, fragments of such proteins or peptides, and antibodies including antibody fragments. In one embodiment, where the agent is a peptide growth factor or cytokine, then it must be a molecule that can be secreted from the effector cell once it is recombinantly expressed by the effector cell.

The term “enzymes” as used herein includes, but is not limited to, enzymes that are capable of synthesizing small molecules like retinoic acid and other steroids (where the ultimate agent to which the target cell will be exposed will be the small molecule produced by the enzyme and secreted from the effector cells) as well as enzymes that are capable of degrading proteins or small molecules such as enzymes of the cytochrome P450 family that are capable of degrading retinoic acid or the enzyme DPP- that is capable of degrading numerous peptides including glucagon-like peptide 1. In one embodiment, where the agent is an enzyme that is capable of synthesizing small molecules in the effector cell, then the enzyme may be a protein that is not secreted from the effector cells upon recombinant expression in the effector cells

By “artificially derived molecules” is meant molecules that are not found in vivo in a biological system but rather are produced synthetically or by recombinant means and that mimic the function of natural molecules.

Examples of such molecules include chimeras of portions of two or more different proteins or peptides as well as analogs or derivatives of naturally occurring proteins or peptides.

By “analogs” is meant a polypeptide wherein one or more amino acid residues of the naturally occurring polypeptide have been substituted by other amino acid residues and/or wherein one or more amino acid residues have been deleted from the polypeptide and/or wherein one or more amino acid residues have been added to the polypeptide. In one embodiment, the addition or deletion of amino acid residues can take place at the N-terminal of the peptide and/or at the C-terminal of the peptide. In another embodiment, 5 or fewer amino acids of the naturally occurring polypeptide are substituted by another amino acid and/or deleted and/or added to the naturally occurring polypeptide. In yet another embodiment 3 or fewer amino acids of the naturally occurring polypeptide are substituted by another amino acid and/or deleted and/or added to the naturally occurring polypeptide.

By “derivative” is meant a chemically modified peptide or an analog thereof, wherein at least one substituent is not present in the unmodified peptide or an analog thereof, i.e. a peptide which has been covalently modified where such modifications include but are not limited to amides, carbohydrates, alkyl groups, acyl groups, esters and the like.

Of course, it is understood that an agent may by itself modulate the biological activity by or in the target cell or it may only modulate the biological activity when it is presented to the target cell in the presence of other agents. In another embodiment, the agent may induce (or suppress) a biological activity by or in the target cell when present at one concentration and suppress (or induce) the same biological activity when present at a different concentration.

In practicing the methods of the invention, it is also understood that different cells may respond to different agents and that different cells may respond to the same agent with an increase or decrease in the same or different biological activities. Thus, the choice of agent or agents to be utilized in the methods of the invention will depend on the target cell that is to be utilized in the methods and the biological activity of the target cell that is to be measured.

For example, if one desires to modulate a chemotactic response of a target cell such as neutrophils or the proliferation of a target cell such as beta cells, then exposure of the target cells to just a single agent may be sufficient to modulate such a biological activity. Alternatively, if the target cells are stem cells and one is seeking to modulate their ability to differentiate into more specialized cells of a desired lineage, then a combination of agents may be more desired in order to modulate the differentiation of the chosen target cell to a cell of desired lineage.

In one embodiment, where the target cells are embryonic stem cells and the biological activity to be modulated is differentiation of the ES cells to cells derived from the endoderm, mesoderm or ectoderm, the choice of agents to use in the methods of the invention will depend on which desired cell lineage one desires to commit the target ES cells to.

For example, for differentiation of ES cells to the endoderm, the agents to be used may be selected from a group of agents that includes but is not limited to, members of the TGFbeta superfamily (particularly Nodal, BMP4, and activin), the Wnt family (particularly Wnt 3 and Wnt 8), the Notch ligands Delta like-1 and Jagged-1 (aka Serrate-1) and the fibroblast growth factor (FGF), epidermal growth factor (EGF) and Hedgehog families. Also, retinoic acid (RA) may be used an agent and this could be applied by having an effector cell express RALDH2 which is the enzyme that synthesizes RA (in this case a “sink” might also be used which would be a population of effector cells expressing CYP26, an RA degrading enzyme). In one embodiment, for the TGFbeta and Wnt families, one could use secreted inhibitors as “sinks” e.g. Chordin, Noggin, Follistatin, Cerberus, Lefty for the TGFbeta and sFrp's and Dkk's for Wnt's.

For differentiation of ES cells to mesoderm and ectoderm the list of agents to be used is essentially the same as for differentiation of ES cells to endoderm but for differentiation to ectoderm one may stimulate with Hedgehog after having neutralized the ES cells by treatment with RA and possibly also Wnt inhibitors. In case of previously neutralized ES cells, Sonic hedgehog (Shh) and Chordin, Noggin may be used to ventralize neural cells and BMP7 to dorsalize the cells.

The effector cells may be transfected with nucleic acid molecules encoding the agent(s) by the methods and means described above for transfection of effector cells by nucleic acid molecules encoding a cell adhesion molecule. In one embodiment, internal ribosome entry sites (IRES) may be used to facilitate expression of a cell adhesion molecule and an agent from a single nucleic acid molecule. For example, one could design a construct which contains in 5′ to 3′ order, a gene encoding a cell adhesion molecule, an IRES and a gene encoding an agent of interest. In an alternative embodiment, one could design a construct which contains in 5′ to 3′ order, a gene encoding a cell adhesion molecule, an IRES and a gene encoding a fluorescent marker protein. An example of such a construct is shown in FIG. 3.

It is also to be understood that in practicing the methods of the invention, the type of assays that will be used to determine or measure the effect of an agent(s) on the biological activity of the target cell will depend on, and vary with, the biological activity that is to be measured.

For example, if one is using the methods of the invention to stimulate the differentiation of ES cells to pancreatic beta cells, one might assay for the production of beta cells by looking for the presence of certain markers in cells of the cell culture (for example, for beta cells, it is insulin+NR×6.1+PDX+Hl×B9+GLUT2+) where the types of assays one could use to measure such markers include, but are not limited to, mRNA detection (e.g. RT-PCR, Northerns, RNAse protection and microarrays) and immunodetection assays. For example, one could do a microarray comparison between natural beta cells and the ES derived beta cells to demonstrate that they are expressing the same set of genes.

Alternatively, if one is using the methods of the invention to stimulate the differentiation of ES cells to pancreatic beta cells, one could assay for the ability of cells in the culture to secrete insulin in response to appropriate stimuli such as glucose and certain amino acids such as arginine where examples of assays useful for measuring insulin release include ELISA and RIA.

Of course, if the assay utilized in the methods of the invention fails to detect any modulation of the biological activity of the target cell by the agent(s) expressed in the effector cells, one of ordinary skill in the art would recognize that there are numerous tests one could conduct to determine whether the negative result is actually due to the agent(s) not being able to modulate the biological activity.

For example, if the target cells had been exposed to multiple agents, one could test the effect of each agent individually on the biological activity of the target cells. In another embodiment, one could confirm that the agent expressed in the effector cells is “active” by placing the effector cells into a system that has been previously demonstrated to accurately measure the activity of the agent. In yet another embodiment, one could transfect the target cells with a reporter gene responsive to the agent being tested in order to determine that the effector cells are expressing the agent in an amount and in a form that is biologically active. The method of the present invention for modulating the biological activity of a target cell may also be used sequentially with two different populations of effector cells expressing different agent(s). For example, one could expose target cells such as ES cells to a population of effector cells transfected with nucleic acid sequence encoding growth factors A and B to produce cell A and then replace that first population of effector cells with a second population of effector cells transfected with nucleic acid sequence encoding growth factors C and D to stimulate differentiation of cell A to cell B. In one embodiment, one can remove the first population of effector cells from the cell culture by transfecting the first population of effector cells with a nucleic acid sequence that encodes a molecule that permits the transfected cells to be eliminated by treatment with a selection agent. An example of such an approach would be transfection of the first population of effector cells with a sequence encoding herpes simplex thymidine kinase and then treating the transfected cells with gancyclovir (i.e. a “selection agent”) to eliminate them. In another embodiment, a suitable selection system could be an inducible Diptheria toxin A-chain construct for example regulated by tetracycline/doxycycline.

Of course, it will be recognized that such an approach can be used even when the methods of the present invention are not to be used sequentially since it may be desirable to eliminate effector cells from the cell culture after the biological activity of the target cells has been modulated. For example, in the methods of the invention, if one exposed target cells to effector cells transfected with sequence encoding growth factors A and B (the effector cells also being transfected with sequence encoding at least one cell adhesion molecule and optionally a fluorescent marker protein), one could also transfect the effector cells with a nucleic acid sequence that encodes a molecule that permits the transfected cells to be eliminated by treatment with a selection agent such that one could then expose the cell culture to the selection agent to eliminate the effector cells.

In an alternative embodiment, one may selectively eliminate the effector cells from the cell culture of effector cells and target cells by transfecting the target cells with a sequence that encodes an antibiotic resistance gene such that treatment of the cell culture with the appropriate antibiotic would eliminate the effector cells but not the transfected target cells For example, transfection of insulin promoter-NeoR into target cells could be used to eliminate non-beta cells.

In culturing the target cells and effector cells of the present invention, the medium one uses may be any suitable medium in which the cells are typically cultured. In one embodiment, the medium used will be a culture medium that the target cells are typically cultured in.

Suitable support surfaces for targeting the target and effector cells of the invention include, but are not limited to, ceramic, metal, glass or polymer surfaces where such surfaces may be in the form of vessels including, but not limited to, plastic dishes, flasks and microtiter plates.

The present invention also relates to target cells that have been exposed to agents according to the methods of the invention, to cells that have been generated from target cells exposed to agents according to the methods of the invention, to pharmaceutical compositions comprising such cells, and to method of use of such cells.

The present invention further provides a method of producing a continuous concentration gradient of one or more agents of interest in a cell culture where the method comprises culturing effector cells that recombinantly express at least one cell adhesion molecule and one or more agents of interest for a time sufficient to allow the cells expressing the cell adhesion molecule(s) to form self-assembling aggregates and secrete the recombinantly expressed agent(s) thereby producing a continuous concentration gradient of the agent(s).

All scientific publications and patents cited herein are specifically incorporated by reference.

1. A method for modulating a biological activity of target cells, said method comprising:
a) exposing a cell culture containing the target cells to effector cells that recombinantly express at least one cell adhesion molecule and at least one agent where expression of the agent(s) by the effector cells produces a concentration gradient of an agent(s) in the cell culture medium; and
b) culturing the target cells in the presence of said effector cells for a time sufficient to permit the biological activity of said target cells to be modulated by said agent(s) in the cell culture medium.

2. The method of claim 1, further comprising as step c) detecting or measuring the effect of said agent(s) on said biological activity of said target cells.

3. The method of claim 1, wherein the cell adhesion molecule recombinantly expressed by the effector cells is selected from a member of the cadherin superfamily.

4. The method of claim 3, wherein the member of the cadherin superfamily is selected from the group consisting of N-cadherin, E-cadherin, R-cadherin, P-cadherin and B-cadherin.

5. The method of claim 1, wherein the effector cells exhibit low endogenous expression of the cell adhesion molecule recombinantly expressed by the effector cells.

6. The method of claim 1, wherein in step a) there are at least two populations of effector cells in the cell culture, a first population of effector cells that recombinantly express a first cell adhesion molecule and a second population of effector cells that recombinantly express a second cell adhesion molecule.

7. The method of claim 6, wherein the first population of effector cells has been transfected with a nucleic acid sequence encoding a first fluorescent marker protein and the second population of effector cells has been transfected with a nucleic acid sequence encoding a second fluorescent marker protein.

8. The method of claim 1, wherein the effector cells have been transfected with a nucleic acid sequence that encodes a molecule that permits the transfected cells to be eliminated by treatment with a selection agent.

9. The method of claim 1, wherein the agent(s) recombinantly expressed by the effector cells is the same molecule(s) as the agent(s) in the cell culture medium.

10. The method of claim 8, wherein the agent(s) recombinantly expressed by the effector cells is secreted from the effector cells into the cell culture medium.

11. The method of claim 1, wherein the agent(s) recombinantly expressed by the effector cells and the agent(s) in the cell culture medium are different molecules.

12. The method of claim 10, wherein the agent(s) recombinantly expressed in the effector cells is an enzyme that produces a molecule in the effector cells that upon secretion from the effector cells is the agent(s) in the cell culture medium.

13. The method of claim 1, wherein said biological activity of said target cells is proliferation of said target cells.

14. The method of claim 12, wherein said target cells are pancreatic beta cells.

15. The method of claim 12, wherein the modulation of said proliferation is an increase in said proliferation.

16. The method of claim 12, wherein the modulation of said proliferation is a decrease in said proliferation.

17. The method of claim 12, wherein the agent recombinantly expressed by the effector cells is a single polypeptide.

18. The method of claim 16, wherein the recombinantly expressed polypeptide is secreted from the effector cells into the cell culture medium.

19. The method of claim 1, wherein the biological activity of said target cells is chemotaxis.

20. The method of claim 18, wherein said target cells are monocytes, neutrophils or macrophages.

21. The method of claim 18, wherein the agent recombinantly expressed by the effector cells is a single polypeptide.

22. The method of claim 20, wherein the recombinantly expressed polypeptide is secreted from the effector cells into the cell culture medium.

23. The method of claim 1, wherein said biological activity of said target cells is differentiation of said target cells to cells of a desired lineage.

24. The method of claim 22, wherein said target cells are stem cells.

25. The method of claim 24, wherein said stem cells are embryonic stem cells.

26. The method of claim 24, wherein said stem cells are adult stem cells.

27. The method of claim 24, further comprising as step c) detecting or measuring the effect of said agent(s) on the differentiation of said stem cells to cells of a desired lineage.

28. The method of claim 27, wherein the effect of said agent(s) on the differentiation of said stem cells is detected or measured in step c) by the expression on or by said cells of a desired lineage of a marker of said cells of a desired lineage.

29. The method of claim 24, wherein said agent(s) induce or promote the differentiation of said embryonic stem cells to endoderm.

30. The method of claim 24, wherein said agent(s) induce or promote the differentiation of said embryonic stem cells to mesoderm.

31. The method of claim 24, wherein said agent(s) induce or promote the differentiation of said embryonic stem cells to ectoderm.

32. The method of claim 24, wherein said stem cells are hematopoietic stem cells.

33. The method of claim 24, wherein said stem cells are bone-marrow derived stem cells.

34. The method of claim 24, wherein said stem cells are intestinal stem cells.

35. The method of claim 34, wherein the cells of a desired lineage that said intestinal stem cells differentiate into are pancreatic cells, preferably endocrine cells.

36. The method of claim 1, wherein said effector cells recombinantly expressing a cell adhesion molecule form cellular aggregates in said cell culture with other effector cells that recombinantly express the same cell adhesion molecule.

37. The method of claim 1, further comprising after step b), removing from said cell culture the effector cells of step a).

38. The method of claim 37, wherein said effector cells are removed from the cell culture by the introduction to the cell culture of a selection agent that selectively eliminates the effector cells.

39. The method of claim 38, wherein said effector cells had been transfected with a nucleic acid sequence that encodes a molecule that permits the transfected cells to be eliminated by treatment with the selection agent.

40. A method for evaluating a candidate agent for its ability to modulate a biological activity of target cells, said method comprising:
a) exposing a cell culture containing the target cells to effector cells that recombinantly express at least one cell adhesion molecule and at least one candidate agent where expression of the candidate agent(s) by the effector cells produces a concentration gradient of candidate agent(s) in the cell culture medium; and
b) detecting or measuring the biological activity of said target cells, wherein the detection or measurement of a change of the biological activity of the target cells in the presence of the candidate agent(s) indicates that the candidate agent(s) in the cell culture medium is capable of modulating the biological activity of the target cells.

41. The method of claim 39, wherein the change in the biological activity of the target cells in the presence of the candidate agent(s) in the cell culture medium is determined by comparing the biological activity of the target cells in the presence of the candidate agent(s) to the biological activity of the target cells in the absence of the candidate agent(s).

42. A method for stimulating the differentiation of embryonic stem cells to pancreatic progenitor cells, said method comprising:
a) exposing a cell culture containing the embryonic stem cells to effector cells that recombinantly express at least one cell adhesion molecule and at least one agent where expression of the agent(s) by the effector cells produces a concentration gradient of agent(s) in the cell culture medium; and
b) culturing the embryonic stem cells in the presence of said effector cells for a time sufficient to permit the differentiation of embryonic stem cells to pancreatic progenitor cells.

43. The method of claim 42, further comprising as step c), detecting or measuring the differentiation of the embryonic stem cells to pancreatic progenitor cells.

44. The method of claim 43, wherein the differentiation of embryonic stem cells to pancreatic progenitor cells is detected or measured by detection of a marker specific for the pancreatic progenitor cells in said cell culture.

45. The method of claim 41, wherein the effector cells are fibroblasts.

46. The method of claim 41, wherein the cell adhesion molecule recombinantly expressed by the effector cells is selected from the group consisting of N-cadherin, E-cadherin, R-cadherin, P-cadherin and B-cadherin.

47. A method for stimulating the differentiation of pancreatic progenitor cells to beta cells, said method comprising:
a) exposing a cell culture containing the pancreatic progenitor cells to effector cells that recombinantly express at least one cell adhesion molecule and at least one agent where expression of the agent(s) by the effector cells produces a concentration gradient of agent(s) in the cell culture medium; and
b) culturing the pancreatic progenitor cells in the presence of said effector cells for a time sufficient to permit the differentiation of pancreatic progenitor cells to beta cells.

48. The method of claim 46 further comprising as step c) detecting or measuring the differentiation of the pancreatic progenitor cells to beta cells.

49. The method of claim 48, wherein the differentiation of pancreatic progenitor cells to beta cells is measured by the detection of insulin production in the cell culture.

50. The method of claim 48, wherein the differentiation of pancreatic progenitor cells to beta cells is measured by the detection of glucose responsive cells in the cell culture.

51. The method of claim 48, wherein the differentiation of pancreatic progenitor cells to beta cells is measured by the detection of a marker specific for beta cells in the cell culture.

52. A method for producing an isolated population of pancreatic progenitor cells from embryonic stem cells, said method comprising:
a) stimulating the differentiation of embryonic stem cells to pancreatic progenitor cells according to the method of claim 41; and
b) isolating said pancreatic progenitor cells.

53. A method for producing an isolated population of beta cells from pancreatic progenitor cells, said method comprising:
a) stimulating the differentiation of pancreatic progenitor cells to beta cells according to the method of claim 46; and
b) isolating said beta cells.

54. Isolated pancreatic progenitor cells produced according to the method of claim 52.

55. Isolated beta cells produced according to the method of claim 53.

56. A pharmaceutical composition comprising the pancreatic progenitor cells of claim 54.

57. A pharmaceutical composition comprising the beta cells of claim 55.

58. A method for treating type 1 diabetes, said method comprising administering to a subject in need of such treatment an effective amount of the pharmaceutical composition of claim 57.

59. A method for producing an isolated population of target cells whose biological activity has been modulated according to the method of claim 1, said method comprising:
a) treating the cell culture of effector cells and target cells with a selection agent that selectively eliminates the effector cells; and
b) isolating said target cells from the treated cell culture

60. Isolated target cells produced according to the method of claim 59.

61. A method of producing a continuous concentration gradient of one or more agents of interest in a cell culture, said method comprising culturing a cell culture of effector cells that recombinantly express at least one cell adhesion molecule and one or more agents of interest under conditions and for a time sufficient to allow the effector cells recombinantly expressing the cell adhesion molecule(s) to form self-assembling aggregates and secrete the recombinantly expressed agent(s) into the cell culture medium thereby producing a continuous concentration gradient of the agent(s) in the cell culture medium.

62. A method for stimulating the differentiation of embryonic stem cells to epiblast cells, said method comprising:
a) exposing a cell culture containing the embryonic stem cells to effector cells that recombinantly express at least one cell adhesion molecule and at least one agent where expression of the agent(s) by the effector cells produces a concentration gradient of agent(s) in the cell culture medium; and
b) culturing the embryonic stem cells in the presence of said effector cells for a time sufficient to permit the differentiation of embryonic stem cells to epiblast cells.

63. A method for stimulating the differentiation of epiblast cells to endoderm cells, said method comprising:
a) exposing a cell culture containing the epiblast cells to effector cells that recombinantly express at least one cell adhesion molecule and at least one agent where expression of the agent(s) by the effector cells produces a concentration gradient of agent(s) in the cell culture medium; and
b) culturing the epiblast cells in the presence of said effector cells for a time sufficient to permit the differentiation of epiblast cells to endoderm cells.

64. A method for stimulating the differentiation of endoderm cells to pancreatic endoderm cells, said method comprising:
a) exposing a cell culture containing the endoderm cells to effector cells that recombinantly express at least one cell adhesion molecule and at least one agent where expression of the agent(s) by the effector cells produces a concentration gradient of agent(s) in the cell culture medium; and
b) culturing the embryonic stem cells in the presence of said effector cells for a time sufficient to permit the differentiation of endoderm cells to pancreatic endoderm cells.

65. A method for stimulating the differentiation of pancreatic endoderm cells to endocrine cells, said method comprising:
a) exposing a cell culture containing the pancreatic endoderm cells to effector cells that recombinantly express at least one cell adhesion molecule and at least one agent where expression of the agent(s) by the effector cells produces a concentration gradient of agent(s) in the cell culture medium; and
b) culturing the embryonic stem cells in the presence of said effector cells for a time sufficient to permit the differentiation of pancreatic endoderm cells to endocrine cells.

66. A method for stimulating the differentiation of endocrine cells to beta cells, said method comprising:
a) exposing a cell culture containing the endocrine cells to effector cells that recombinantly express at least one cell adhesion molecule and at least one agent where expression of the agent(s) by the effector cells produces a concentration gradient of agent(s) in the cell culture medium; and
b) culturing the embryonic stem cells in the presence of said effector cells for a time sufficient to permit the differentiation of endocrine cells to beta cells.
